(19) United States
(12) Patent Application Publication
(10) Pub. No.: US 2022/0072076 A1
(43) Pub. Date: Mar. 10, 2022

(54) Therapeutic Mixture with Oregano Oil for Herpes Infections

(71) Applicants: Mantsur Yerushalmi, Herzelia (IL); Sigal Yerushalmi, Herzelia (IL)

(72) Inventors: Mantsur Yerushalmi, Herzelia (IL); Sigal Yerushalmi, Herzelia (IL)

(21) Appl. No.: 17/467,319

(22) Filed: Sep. 6, 2021

(30) Foreign Application Priority Data
Sep. 9, 2020 (IL) 277230

(51) Int. Cl. A61K 36/53; A61Q 19/10; A61K 8/9789

(52) U.S. Cl. CPC A61K 36/53; A61K 8/9789; A61Q 19/10

(57) ABSTRACT

A topically therapeutic mixture for treatment of infections and diseases of herpes viruses that includes essential oregano oil, carrier oil and basic gel. The essential oregano oil is in the amount of about 7% by weight of the mixture, the carrier oil is in the amount of about 15% by weight of the mixture, and the basic gel is in the amount of about 78% by weight of the mixture. A method for treatment of that infections and diseases by applying the therapeutic mixture on the infected area or on the back along the spine or by providing a soap or a body wash that includes essential oregano oil in the amount of about one Per-mille by weight and soaping the body.

### TECHNICAL FIELD

The present invention relates to a therapeutic mixture for the treatment of infections and diseases in humans that are caused by herpes viruses that comprises essential oregano oil, carrier oil, and gel.

### BACKGROUND ART

There is a real need for an effective treatment for herpes viruses as well as for the infections and diseases of these viruses. The present invention discloses a method and a therapeutic mixture for such treatments.

### DESCRIPTION OF THE DRAWINGS

The intention of the drawings attached to the application is not to limit the scope of the invention and its application. The drawings are intended only to illustrate the invention and they constitute only one of its many possible implementations.

FIG. 1 schematically illustrates the therapeutic mixture (1) that includes essential oregano oil (2), carrier oil (3), and a basic gel (4).

### THE INVENTION

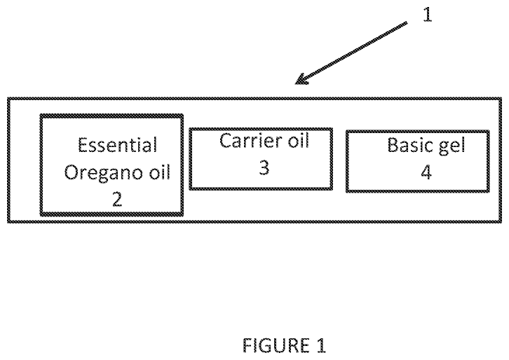

The present invention is based on the use of oregano oil in the treatment of herpes viruses, and therefore the main object of the present invention is to provide a therapeutic mixture for the treatment of herpes viruses. The therapeutic mixture (1) should be used topically for the treatment of infections and diseases in humans that are caused by herpes viruses and that include essential oregano oil (2), carrier oil (3), and a basic gel (4). The mixture may include also vitamin E oil and any other useful material for skin. Vitamin E Oil is repairing and moisturizing oil and an antioxidant and suits as an addition to skin products. The essential oregano oil, the carrier oil, and the basic gel can be purchased in the market as ready to be mixed products. FIG. 1 schematically illustrates the therapeutic mixture (1) that includes essential oregano oil (2), carrier oil (3), and a basic gel (4).

The herpes viruses that are also known as Herpesviridae are a family of DNA viruses that cause infections and certain diseases in animals and humans. This group of viruses includes the HHV1 that causes sores and blisters on the lips, HHV2 that causes sores and blisters in the genital area, and HHV3 that causes a Herpes Zoster. The term “treatment” is used conventionally and refers to the purpose to diminish the symptoms of these viruses, significant decrease in the incidence of onset of symptoms and also for the disappearance of the viruses.

The preparation of the mixture can be as it is customary to prepare cosmetic mixtures, in which it is customary to use a basic gel that enables an effective and easy use and application of the mixture on the skin, a carrier oil to which the main oil it carries (in our case the oregano oil) adheres as it penetrates through the skin. The main purpose is to infuse the oregano oil under the skin and for that we should use the mixture that enables us to do that effectively.

The term essential oil is known to professionals who act in this field and in a nutshell we can say the following: Essential oils are compounds extracted from plants that capture the plant's scent and flavor, or “essence”. Unique aromatic compounds give each essential oil its characteristic essence. Essential oils are obtained through distillation (via steam and/or water) or mechanical methods, such as cold pressing. By “essential oil” is meant the concentrated liquid of the volatile compounds of a plant.

The term carrier oil is known to professionals who act in this field and in a nutshell we can say the following: Essential oils are distilled from plants and if applied to the skin directly, they can cause reactions, such as severe irritation, redness or burning. Carrier oils are used to dilute the essential oils and help “carry” them into the skin. It is possible to use even a basic gel as carrier oil and Aloe Vera gels and unscented body lotion are also sometimes can be used as carriers and in that case the mixture does not include the carrier oil.

As stated above, the therapeutic mixture should be used topically and is for treating infections and diseases in humans that are caused by herpes viruses, and this mixture comprises essential oregano oil, carrier oil, and basic gel. The therapeutic mixture is highly effective when the essential oregano oil is in the amount of about 7% by weight of the mixture, the carrier oil is in the amount of about 15% by weight of the mixture, and the basic gel is in the amount of about 78% by weight of the mixture.

The present invention also relates to the method of treating infections and diseases in humans that are caused by herpes viruses by providing the therapeutic mixture and applying it on the infected area or on the back along the spine. In addition to that, the present invention also relates to the method of treating infections and diseases that are caused by herpes viruses by providing soap or a body wash that includes essential oregano oil in the amount of about one Per-mille by weight of the soap or body wash, and soaping the body with the soap or the body wash.

Many patients that have suffered from recurrent symptoms of herpes received treatment using the therapeutic mixture. For example, a large number of patients who suffered from severe symptoms of herpes about three to four times a year applied the mixture on the back along the spine once a day for about two weeks, and the symptoms returned only after about two years, and after further similar treatment for about two weeks the symptoms stopped completely. Patients that have suffered from recurrent light symptoms of herpes (usually on their lips) about every month applied the mixture for a week or so and the symptoms now returned once a year or two.

It is possible and even recommended to use adhesive gel pads (are known also as gel plasters) as a way to deliver the therapeutic mixture to the patient. This is a known method in which the pad soaked in the therapeutic mixture and the pad also includes a sticky material that enables the user to attach the pad to the skin for an extended period of time, several hours or an entire night, during which the mixture penetrates under the skin.

What we claim is:

1. A therapeutic mixture to be used topically for the treatment of infections and diseases that are caused by herpes viruses, comprising: essential oregano oil, and basic gel.

2. The therapeutic mixture of claim 1 that further includes carrier oil.

3. The therapeutic mixture of claim 1, wherein said essential oregano oil is in the amount of about 7% by weight of the mixture.

4. The therapeutic mixture of claim 2, wherein said essential oregano oil is in the amount of about 7% by weight of the mixture, the carrier oil is in the amount of about 15% by weight of the mixture, and the basic gel is in the amount of about 78% by weight of the mixture.

5. A method for treatment of infections and diseases that are caused by herpes viruses, comprising:
(a) providing a therapeutic mixture of essential oregano oil and basic gel or of essential oregano oil, carrier oil and basic gel; and
(b) applying the therapeutic mixture on the infected area or on the back along the spine.

6. A method for treatment of infections and diseases that are caused by herpes viruses, comprising:
(a) providing a soap or a body wash that includes essential oregano oil in the amount of about one Per-mille by weight of the soap or the body wash; and
(b) soaping the body with said soap or a body wash.
